(19) United States
(12) Patent Application Publication
(10) Pub. No.: US 2010/0021504 A1
(43) Pub. Date: Jan. 28, 2010

(54) TREATMENT AND PREVENTION OF ALLERGIC AIRWAYS DISEASES

(75) Inventors: Peter Gibson, Merewether (AU); Philip Hansbro, The Hill (AU)

Correspondence Address: MERCHANT & GOULD PC, P.O. BOX 2903, MINNEAPOLIS, MN, 55402-0903, US

(73) Assignee: NEWCASTLE INNOVATION LIMITED, Callaghan, New South Wales (AU)

(21) Appl. No.: 12/376,032

(22) PCT Filed: Aug. 3, 2007
(86) PCT No.: PCT/AU2007/001098

§ 371 Date: Oct. 9, 2009

(30) Foreign Application Priority Data
Aug. 3, 2006 (AU) 2006904309

(51) Int. Cl. A61K 39/09; A61K 35/74; A61P 11/06

(52) U.S. Cl. 424/244.1; 424/93.44

(57) ABSTRACT

The present invention provides a method for the treatment or prevention of an allergic airways disease in a subject, the method comprising administering to the subject a composition comprising Streptococcus pneumoniae or one or more antigens derived therefrom. Also provided are methods and compositions for inducing protective immunity against an allergic airways disease, suppressing an allergic immune response, and treating or preventing conditions such as eosinophilia, mucous secreting cell expression, airway hyperresponsiveness and Th2-mediated disease.

### FIELD OF THE INVENTION

The present invention relates generally to methods for the treatment and prevention of allergic airways diseases such as asthma. More particularly the invention relates to the immunization of individuals using vaccines and/or therapies based on Streptococcus pneumoniae and to the use of such vaccines and/or therapies for the treatment or prevention of allergic airways diseases.

### BACKGROUND OF THE INVENTION

Allergic airways diseases such as asthma and allergic rhinitis are of major, and increasing, public health concern, especially in industrialised nations where they represent the most common chronic disorders in children. Asthma, in particular, is a chronic respiratory disorder that has increased alarmingly in prevalence in the last 20 years. Australia has one of the highest rates of asthma in the world, with estimates suggesting that up to 25-30% of the population are affected. In addition to being potentially debilitating for sufferers, the direct and indirect costs of allergic airways diseases on health systems are substantial.

There is a clear need not only for effective therapies for the treatment and management of allergic airways diseases, but also for strategies and approaches to prevent the onset and development of such diseases.

Asthma is an inflammatory disorder causing variability of airflow obstruction, and an increased sensitivity and exaggerated response to many different stimuli (airway hyperresponsiveness), particularly allergens. Together these patho-physiological manifestations lead to symptoms including wheezing, coughing, chest tightness and dyspnoea. The chronic inflammatory response in asthma is characterised by an intense eosinophil infiltrate into the airways and mucous secreting cell hyperplasia that is coordinated by cytokine release from T-helper type 2 (Th2) lymphocytes. Eosinophils release a range of both preformed and newly synthesised mediators that damage the mucosal epithelial lining and promote an exaggerated repair response resulting in tissue remodeling and sub-epithelial fibrosis.

It has been noted that the prevalence of asthma varies inversely with the prevalence of certain bacterial infections such as tuberculosis and typhoid (see for example Jones et al., 2000). These infections, together with those of Mycobacterium bovis elicit a T-helper type 1 (Th1) immune response, which, during the early years of life, may cause immune deviation from the neonatal Th2 response to a mature Th1 response. The absence of exposure to Th1 inducing infections is thought to promote the persistence of a Th2 phenotype and permits the development of allergy and asthma.

The increasing prevalence of allergic airways diseases, and the increasingly early onset of diseases such as asthma in children, has focused much attention on the need for effective treatments and preventative measures. Whilst research continues, to date there has yet to emerge any effective means of preventing the onset or development of allergic airways diseases such as asthma.

The present invention is predicated on the inventors surprising finding that in a mouse model of ovalbumin-induced allergic airways disease, immunization with killed Streptococcus pneumoniae bacteria reduced eosinophilia and airways hyperresponsiveness.

### SUMMARY OF THE INVENTION

According to a first aspect of the present invention there is provided a method for the treatment or prevention of an allergic airways disease in a subject, the method comprising administering to the subject a composition comprising Streptococcus pneumoniae or one or more antigens derived therefrom.

The composition may be in the form of a vaccine and the administration may comprise vaccination. The vaccine may be a therapeutic or prophylactic vaccine.

The composition or vaccine may comprise killed or attenuated Streptococcus pneumoniae. The Streptococcus pneumoniae may be of any serotype or strain. In one embodiment the strain is NC012695.

The one or more antigens may comprise cellular fractions, components or constituents. Exemplary antigenic constituents include, but are not limited to, cell wall peptidoglycan, lipoteichoic acid, cell wall polysaccharides and proteins, capsular polysaccharides and proteins such as pneumolysin, PsaA, PspA and CbpA, lipids, carbohydrates, glycoproteins, and fragments thereof.

The allergic airways disease may be selected from asthma, asthma exacerbations, eosinophilic bronchitis, allergic rhinitis, chronic cough, sinusitis, angioedema, urticaria, chronic obstructive pulmonary disease, conjunctivitis and hay fever. The asthma may be chronic or acute.

The vaccination or therapy may result in the inducement of an innate and/or adaptive immune response. Accordingly, typically the method comprises administering an immunologically effective amount of Streptococcus pneumoniae or one or more antigens derived therefrom.

According to a second aspect of the present invention there is provided a method for inducing protective immunity against an allergic airways disease in a subject, the method comprising administering to the subject a composition comprising Streptococcus pneumoniae or one or more antigens derived therefrom.

The composition may be in the form of a vaccine and the administration may comprise vaccination.

According to a third aspect of the present invention there is provided a method for the suppression of an allergic immune response in a subject, the method comprising administering to the subject a composition comprising Streptococcus pneumoniae or one or more antigens derived therefrom.

The composition may be in the form of a vaccine and the administration may comprise vaccination.

The allergic immune response may be a response of the innate or adaptive immune system. The immune response may be a Th2 immune response.

The allergic immune response may be associated with, for example, eosinophilia, mucous secreting cell expression and/or airway hyperresponsiveness. The eosinophilia may be peripheral or tissue eosinophilia.

According to a fourth aspect of the present invention there is provided a method for the treatment or prevention of one or more of eosinophilia, mucous secreting cell expression, airway hyperresponsiveness and/or Th2-mediated disease in a subject, the method comprising administering to the subject a composition comprising Streptococcus pneumoniae or one or more antigens derived therefrom.

The composition may be in the form of a vaccine and the administration may comprise vaccination.

The eosinophilia may be peripheral or tissue eosinophilia.

According to a fifth aspect of the present invention there is provided a composition or vaccine for use in the treatment or prevention of allergic airways diseases, eosinophilia, mucous secreting cell expression, airway hyperresponsiveness or Th2-mediated disease, the composition or vaccine comprising Streptococcus pneumoniae or one or more antigens derived therefrom.

The allergic airways disease may be selected from asthma, asthma exacerbations, eosinophilic bronchitis, allergic rhinitis, chronic cough, sinusitis, angioedema, urticaria, chronic obstructive pulmonary disease, conjunctivitis and hay fever.

According to a sixth aspect of the present invention there is provided a method for the prevention or suppression of onset of an allergic airways disease in a subject, the method comprising administering to the subject a composition comprising Streptococcus pneumoniae or one or more antigens derived therefrom.

The composition may be in the form of a vaccine and the administration may comprise vaccination.

According to a seventh aspect of the present invention there is provided a method for the treatment or prevention of asthma or asthma exacerbations in a subject, the method comprising administering to the subject a composition comprising Streptococcus pneumoniae or one or more antigens derived therefrom.

The composition may be in the form of a vaccine and the administration may comprise vaccination.

According to an eighth aspect of the present invention there is provided a method for the prevention or suppression of onset of an allergic airways disease in a subject, the method comprising administering to the subject a composition comprising whole killed Streptococcus pneumoniae.

The composition may be in the form of a vaccine and the administration may comprise vaccination.

According to a ninth aspect of the present invention there is provided the use of killed or live attenuated Streptococcus pneumoniae or one or more antigens derived therefrom for the manufacture of a composition or vaccine for the treatment or prevention of allergic airways diseases.

In accordance with the aspects and embodiments of the present invention, compositions or vaccines may be administered in conjunction with one or more suitable adjuvants designed to improve the delivery or efficacy of the composition or vaccine, such as for example CpG oligonucleotides. Such suitable adjuvants may be present in the composition or vaccine.

Aspects and embodiments of the present invention are applicable to any organism susceptible to allergic airways diseases. Typically the subject is a mammal. Typically the mammal is a human.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

### BRIEF DESCRIPTION OF THE DRAWINGS

Embodiments of the present invention will now be described, by way of example only, with reference to the accompanying drawings.

FIG. 1. OVA-specific-IL-5 (A) and -IFNγ (B) production from mediastinal lymph node T cells restimulated with OVA and OVA-specific IgG1 serum antibodies (C) in mice infected with Streptococcus pneumoniae before OVA challenge following intraperitoneal OVA sensitisation (Spn+OVA). Results are presented as mean±SEM. n=8-10 for each group. **p<0.01, ***p<0.001 compared to uninfected allergic controls (OVA).

FIG. 2. Blood (A) and BALF (B) eosinophilia in mice infected with Streptococcus pneumoniae before OVA challenge following intraperitoneal OVA sensitisation (Spn+OVA). Results are expressed as mean±SEM. n=8-10 for each group. **p<0.01 compared to uninfected allergic controls (OVA).

FIG. 3. Airways hyperresponsiveness, in terms of lung resistance (A) and dynamic compliance (B), in mice infected with Streptococcus pneumoniae before OVA challenge following intraperitoneal OVA sensitisation (Spn+OVA). Results are presented as mean±SEM. n=8-10 for each group. *p<0.05 for entire dose-response curve compared to uninfected allergic controls (OVA).

FIG. 4. OVA-specific-IL-5 (A) and -IFNγ (B) production in OVA re-stimulated mediastinal lymph node T cells and OVA-specific IgG1 serum antibodies (C) in mice with a resolved or concurrent Streptococcus pneumoniae infection at the time of intraperitoneal OVA sensitisation. Results are presented as mean±SEM. n=8-10 for each group. *p<0.05, **p<0.01, ***p<0.001 compared to uninfected allergic controls (OVA).

FIG. 5. Blood (A), BALF (B), and tissue eosinophilia (C), and goblet cell hyperplasia (D) 1, 4 and 8 days after OVA challenge in mice infected with Streptococcus pneumoniae during intranasal OVA sensitisation. *p<0.05, **p<0.01, ***p<0.001 compared to uninfected allergic controls (OVA).

FIG. 6. Blood (A), BALF (B) and tissue (C) eosinophils 4 days after OVA challenge of mice infected with Streptococcus pneumoniae before, during or after intranasal OVA sensitisation. *p<0.05, **p<0.01, ***p<0.001 compared to uninfected allergic controls (OVA).

FIG. 7. Goblet cell hyperplasia (A) and histopathological score (B) 4 days after OVA challenge of mice infected with Streptococcus pneumoniae before, during or after OVA intranasal sensitisation, **p<0.01 compared to uninfected allergic controls (OVA).

FIG. 8. Airways hyperresponsiveness, in terms of lung resistance (A) and dynamic compliance (B), in mice with a resolved or concurrent Streptococcus pneumoniae infection at the time of intraperitoneal OVA sensitisation. Results are presented as mean±SEM. n=8-10 for each group. *p<0.05 for entire dose response-curve compared to uninfected allergic controls (OVA).

FIG. 9. Schematic representation of experimental protocols for evaluating the affect of Streptococcus pneumoniae immunisation on allergic airways disease. Mice were immunised with whole killed Streptococcus pneumoniae before (A), during (B) or after (C) intraperitoneal OVA sensitisation.

FIG. 10. Mediastinal lymph node cells were collected from mice immunised with whole killed Streptococcus pneumoniae before, during or after intraperitoneal OVA sensitisation and stimulated in vitro with OVA. OVA-specific-IL-5 (A), -IL-13 (B) and -IFN-γ (C) were determined by ELISA. n=8-10 per group. (*p<0.05, **p<0.01, ***p<0.001 compared to un-infected allergic (OVA) controls).

FIG. 11. Blood (A), BALF (B) and peribronchial (C) eosinophilia was assessed mice immunised with whole killed Streptococcus pneumoniae before, during or after intraperitoneal OVA sensitisation (followed by intranasal OVA challenge). n=8-10 per group. (*p<0.05, **p<0.01, ***p<0.001 compared to un-infected allergic (OVA) controls).

FIG. 12. Goblet cell hyperplasia of the large airways was determined in mice immunised with whole killed Streptococcus pneumoniae before, during or after intraperitoneal OVA sensitisation (followed by intranasal OVA challenge). n=8-10 per group. (**p<0.01 compared to un-infected allergic (OVA) controls).

FIG. 13. Airway hyperresponsiveness, in terms of lung resistance (A-C) and dynamic compliance (D-F), in mice immunised with whole killed Streptococcus pneumoniae before, during or after intraperitoneal OVA sensitisation (followed by intranasal OVA challenge). n=8-10 per group. (*p<0.05 for entire dose-response curve compared to un-infected allergic (OVA) controls).

FIG. 14. T cell numbers from mediastinal lymph nodes determined by flow cytometry. Mediastinal lymph node cells were collected from mice infected with Streptococcus pneumoniae concomitant with OVA sensitisation (Spn+OVA) and uninfected mice sensitised with OVA. Cells were cultured in vitro in the presence or absence of OVA prior to staining and analysis. The number of CD4+CD25+ (A), CD4+CD25+Foxp3+ (B) and Foxp3+ Hi (C) cells are shown. ***p<0.001.

FIG. 15. The suppression of allergen specific T cell proliferation by Treg cells from mice infected with Streptococcus pneumoniae concomitant with OVA sensitisation. CD4+ CFSE+ cells were gated via flow cytometry and displayed as a histogram. CD4+CD25− cells alone proliferate (B). When CD4+CD25+ are added, suppression of CD4+ cell proliferation is observed (C).

### DETAILED DESCRIPTION OF THE INVENTION

Streptococcus pneumoniae, a common respiratory pathogen, is the predominant cause of community-acquired pneumonia in children and adults, and frequently induces otitis media, septicaemia and meningitis. Streptococcus pneumoniae vaccination has been recommended to prevent invasive Streptococcus pneumoniae disease in “high risk” groups, including asthmatics (Salisbury and Begg, 1996) and asthma has been suggested as an independent risk factor for invasive Streptococcus pneumoniae disease (Talbot et al., 2005). However Streptococcus pneumoniae infection is not widely implicated in the development and exacerbation of asthma.

As disclosed herein, using a mouse model of Th2-driven allergic airways diseases, the present inventors have demonstrated that not only does Streptococcus pneumoniae infection have a protective effect against development of allergic airways diseases, but surprisingly immunization using whole killed bacteria also suppresses the hallmark features of allergic airways diseases. As exemplified herein, these include inhibition of type 2 cytokine and antibody responses, inhibition of peripheral and tissue eosinophilia, inhibition of goblet cell hyperplasia and inhibition of airways hyperresponsiveness. In particular eosinophilia is linked to the ongoing remodeling of the airways in diseases such as chronic asthma. Without wishing to be bound by theory, as both innate and adaptive immune responses are important in the clearance of, and protection against, Streptococcus pneumoniae infection, either or both the innate and adaptive immune systems may be involved in inhibiting the development and progression of allergic airways diseases such as asthma upon Streptococcus pneumoniae vaccination or therapy in accordance with the invention.

The findings as disclosed herein open up novel and powerful avenues for the treatment and prevention of allergic airways diseases such as asthma based on immunization or therapy using Streptococcus pneumoniae or using fractions, constituents or components of Streptococcus pneumoniae capable of inducing an immune response.

Accordingly, one aspect of the present invention relates to a method for the treatment or prevention of allergic airways disease in a subject, the method comprising administering to the subject a composition comprising Streptococcus pneumoniae or one or more antigens derived therefrom.

The composition may be in the form of a vaccine, and thus the administration of the subject may comprise vaccination of the subject with a vaccine.

The composition or vaccine may comprise live Streptococcus pneumoniae cells, or alternatively may comprise killed cells, or cells otherwise treated such that they are not capable of reproduction in a host. The composition or vaccine, alternatively or in addition, may comprise cell fractions or other antigenic components or constituents of Streptococcus pneumoniae as described herein.

The present invention finds application in the treatment and prevention of a range of allergic airways diseases, including but not limited to asthma, asthma exacerbations, eosinophilic bronchitis, allergic rhinitis, chronic cough, sinusitis, angioedema, urticaria, chronic obstructive pulmonary disease, conjunctivitis and hay fever. In particular, the present invention may be employed in the treatment or prevention of allergic airways diseases associated with a Th2 immune response.

As used herein the term “treatment”, refers to any and all uses which remedy a disease state or one or more symptoms thereof, or otherwise prevent, hinder, retard, or reverse the progression of disease or other undesirable symptoms in any way whatsoever.

As used herein the term “prevention” means generally the prevention of the establishment of an allergic airways disease. In accordance with accepted classification and nomenclature, prevention may be primary, secondary or tertiary (see for example, Carlsen, 2004). Primary prevention refers to the prevention of the establishment of the disease. In one context, this may refer to strategies and approaches adopted at a community level in order to reduce the incidence of the disease, or to an individual level wherein the individual may have no indications of susceptibility to the diseases or of being ‘high risk’. Secondary prevention refers to intervention in individuals who are at high risk for the development of the allergic airways disease and who have not yet developed the disease, but may or may not have exhibited some allergic symptoms. These individuals may have a family history of allergic disease and/or one or more of atopic dermatitis or eczema, food allergy, bronchial hyperreactivity, blood eosinophilia, airway eosinophilia, mucous secreting cell expression, Th2-mediated disease, elevated total IgE levels, elevated allergen-specific IgE, or skin-test reactivity to specific allergens. Tertiary prevention refers to preventing the worsening of the disease and reducing the symptoms experienced by allergic patients.

As used herein the term “immunologically effective amount” refers to the ability of the Streptococcus pneumoniae or antigen derived therefrom to induce an immune response suitable and sufficient to have the desired effect, for example, the treatment or prevention of an allergic airways disease, protecting a subject against an allergic airways disease, and/or treating or preventing eosinophilia, mucous secreting cell expression, airway hyperresponsiveness and/or Th2-mediated disease.

The terms “vaccination” and “vaccinating” mean the inoculation of a substance or composition (a vaccine) into the body of the subject for the purpose of producing immunity against a disease, that is for the purpose of treating or preventing a disease. Accordingly, vaccination may be therapeutic or prophylactic. By therapeutic vaccination is meant the administration of a vaccine to an individual already suffering from an allergic airways disease, typically for the purpose of heightening or broadening the immune response to thereby halt, impede or reverse the progression of the disease. The terms “vaccination” and “immunization” are used interchangeably herein. Similarly, the terms “vaccination” and “administration” are used interchangeably herein in certain contexts.

Vaccination in accordance with the invention may provide protective immunity against allergic airways diseases to the subject being vaccinated. That is, the component(s) of the vaccine may elicit a protective immune response in the subject, for example by inducing the production of autoantibodies, innate immunity or adaptive immunity against the component(s). As used herein, the term “protective immunity” refers to the ability of a molecule or composition administered to a subject to elicit an appropriate immune response in the subject and thereby provide protection to the subject from the development or progression of an allergic airways disease.

The present invention contemplates the vaccination of individuals with vaccines comprising or derived from Streptococcus pneumoniae strains. A typical Streptococcus pneumoniae strain as exemplified herein is NC012695 available under Accession Number ACTC6303 from the National Collection of Type Cultures, Egham, UK. However it will be clear to those skilled in the art that the present invention is not so limited, and any serotype or strain of Streptococcus pneumoniae may be used.

Vaccines for use in accordance with the invention may comprise whole bacteria, or fractions, components or constituents thereof, wherein the fractions, components or constituents are antigens or antigenic in nature. The term “antigen” refers to any substance or product or mixture of substances or products capable of eliciting an immune response (also referred to herein as immunomodulatory agents). Accordingly, the antigen may comprise crude fractions, lysates, purified or partially purified cellular components or constituents or mixtures or combinations of any of the above.

In embodiments in which whole bacteria are administered in accordance with the invention, the bacteria are typically killed or live attenuated such that they are capable of stimulating an immune response in the organism to which they are administered, while causing little or no infection or disease in the organism. For use in accordance with the invention, bacteria may be killed or attenuated by a variety of means well known to those skilled in the art. Where the vaccine comprises killed bacteria, the bacteria may be killed, for example, by heat treatment or using chemical means such as ethanol.

Alternatively, antigenic fractions, components or constituents of Streptococcus pneumoniae may be administered in accordance with the invention. For example, an antigenic fraction may be an extract of whole bacteria produced by any suitable means such as lysis or sonication. An antigenic component or constituent may be an intracellular, extracellular (such as a secreted protein or polypeptide), capsule-associated, cell wall-associated or cell membrane-associated component or a cellular constituent such as a protein, polypeptide, peptide, polysaccharide, carbohydrate, lipid, lipopolysaccharide, glycoprotein, or fragment thereof. An antigenic component or constituent may comprise any two or more of the aforementioned constituents. An antigenic component may be an epitope, for example a peptide sequence, polysaccharide or carbohydrate epitope, or the like, derived from a Streptococcus pneumoniae cellular constituent. The antigen may be natural or modified from its native state, or may be synthetically produced. An antigen suitable for use in accordance with the invention may generate either an adaptive immune response or innate immune response, or both. The immune response may be produced directly by the antigen or indirectly, such as via the inhibition or activation of one or more host factors. For example, the antigen may induce the required immune response via activation of one or more of MyD88, TLR-2 and TLR-4.

By way of non-limiting example only, suitable Streptococcus pneumoniae vaccines suitable for use in accordance with the invention include Pneumovax® 23 (Merck & Co., Inc.) and Prevenar® (PncRM7; also marketed as Prevnar®) (Wyeth). Pneumovax® 23 is a polyvalent vaccine composed of capsular polysaccharides from 23 serotypes of Streptococcus pneumoniae which stimulates a T cell independent immune response generating the production of capsule specific antibodies from B cells. Prevenar is also a multivalent vaccine comprising 7 different Streptococcus pneumoniae polysaccharides conjugated to an immunogenic carrier protein (diphtheria toxoid (CRM197)). This induces specific B cell and T cell responses to the Streptococcus pneumoniae polysaccharides.

Alternatively, specific immunomodulatory agents constructed from Streptococcus pneumoniae components may be employed. Typically, the components selected are known to stimulate strong immune responses that mimic the effects of live or killed Streptococcus pneumoniae administration. These agents may induce both innate and adaptive immune responses. Streptococcus pneumoniae has an outer polysaccharide capsule, which has a typically strong interaction with the host innate immune system during infection. The Streptococcus pneumoniae cell wall contains significant amounts of peptidoglycan and lipoteichoic acid, which are strong TLR-2 stimulatory molecules (see for example Schroder et al., 2003; Dziarski and Gupta, 2005).

Streptococcus pneumoniae cell walls and cells also contain polysaccharides and proteins some of which posses potent adaptive T cell activating capabilities, such as pneumolysin, pneumococcal surface adhesin (Psa)A, Streptococcus pneumoniae surface protein (Psp)A and choline binding protein (Cbp)A. Immunisation with these immunodominant agents confers protection against infection through robust antibody and T cell mediated processes, which is enhanced if proteins are co-administered (see for example Ogunniyi et al., 2000, 2001).

Pneumolysin is one of the most immunogenic proteins of Streptococcus pneumoniae and is critical in protection and in stimulating a strong T cell response and T cell chemotaxis. Pneumolysin also induces immune responses through the activation of MyD88 and both TLR-2 and TLR-4.

Accordingly, by way of non-limiting example only, suitable immunomodulatory agents for use in vaccines in accordance with the present invention include cell wall peptidoglycan, lipoteichoic acid and cell wall polysaccharides and proteins such as pneumolysin, PsaA, PspA and CbpA. Also contemplated herein is the use of mixtures or combinations of any two or more suitable immunomodulatory agents. Such immunomodulatoy agents may similarly be used in conjunction with whole killed or live attentuated bacteria, or with other antigenic fractions, components or constituents. Two or more of said immunomodulatory agents may be conjugated to enhance immunogenicity. For example, an immunogenic Streptococcus pneumoniae polysaccharide may be suitable conjugated with a polypeptide. As exemplified in U.S. Pat. No. 5,565,204 (Kuo et al.), the disclosure of which is incorporated herein in its entirety, an immunogenic capsular polysaccharide may be conjugated with the pneumolysin protein or fragment thereof.

The efficacy of vaccines for use in accordance with the invention may be enhanced by the use of one or more adjuvants. Adjuvants capable of enhancing the delivery or protective or therapeutic efficacy of bacterial vaccines (for example by boosting the immune response produced) are well known to those skilled in the art. For example, the incorporation of bacterial DNA in the form of CpG oligodeoxynucleotides (CpG-ODN) are known to act as strong adjuvants for bacterial vaccines and stimulate Th1 type immune responses (Berry et al., 2004).

Other suitable adjuvants include biodegradable cationic polylactide-co-glycolide (PLG) microparticles, cholera toxin and heat labile enterotoxin. Two or more adjuvants may be used in combination.

Inoculation of a subject with a composition or vaccine in accordance with the invention sufficient to produce the desired therapeutic or prophylactic effect may require only a single administration (or vaccination). In another embodiment of the present invention, the administration regime may comprise a series of administrations to produce a full, broad immune response. For example, when multiple administrations or vaccinations are required, the vaccinations can be provided at suitable intervals depending on the circumstances and the desired outcome; the interval may be hours, days, months or years. For example the interval between administrations or vaccinations may be from about 24 hours to about 6 months; about 24 hours, about 48 hours, about 72 hours, about one week, about two weeks, about one month, about 3 months or about 6 months or longer. Alternatively, it may be appropriate to space administrations or vaccinations over a period of years, such as for example about 1 year, about 2 years, about 3 years, about 4 years, about 5 years, about 10 years or longer. When multiple administrations or vaccinations are required, the doses may or may not be equal doses and similarly when more than two administrations or vaccinations are required the time intervals between individual administrations or vaccinations may or may not be the same. The optimal quantity and spacing of individual dosages will be determined by a variety of factors including the particular disease to be treated or prevented, the form, route and site of administration, and the particular individual being treated. Such optimum conditions can be determined by conventional techniques well known to those skilled in the art.

The effective dose level for any particular patient will depend upon a variety of factors including: the disease to be treated or prevented (and in the case of therapeutic treatment, the severity of the disease), the particular vaccine employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

Those skilled in the art will also readily appreciate that as used herein the terms “effective amount” and “effective dose” include within their meaning a non-toxic but sufficient amount of an agent or compound to provide the desired therapeutic or prophylactic effect. The exact amount required will vary from subject to subject. Thus, it is not possible to specify an exact “effective amount”. One skilled in the art would be able, by routine experimentation, to determine an effective, non-toxic amount of a composition or vaccine which would be required to treat applicable diseases.

By way of example only, an effective dosage may be in the range of about 0.0001 mg to about 1000 mg per kg body weight; about 0.001 mg to about 750 mg per kg body weight; about 0.01 mg to about 500 mg per kg body weight; about 0.1 mg to about 500 mg per kg body weight; about 0.1 mg to about 250 mg per kg body weight; or about 1.0 mg to about 250 mg per kg body weight. Individual dosages of vaccine may comprise about 0.1 to about 5000 μg active component(s), typically about 1 to about 500 μg, about 10 to about 250 μg, about 10 to about 250 μg, about 20 to about 200 μg, about 25 to 100 μg or about 25 to about 50 μg of active components(s).

Routes of administration suitable for methods of the present invention include, but are not limited to, oral (including by inhalation, ingestion or sublingual administration), nasal, topical, parenteral (intramuscular, subcutaneous, intravenous, intraarterial), transmucosal, subcutaneous, transcutaneous and transdermal. Administration may be local, regional or systemic.

In general, suitable compositions may be prepared according to methods which are known to those of ordinary skill in the art and accordingly may include a pharmaceutically acceptable carrier, diluent and/or adjuvant. For administration in accordance with the present invention, a suitable vaccine may be formulated in a pharmaceutically acceptable carrier according to the mode and route of administration to be used. The carriers, diluents and adjuvants must be “acceptable” in terms of being compatible with the other ingredients of the composition, and not deleterious to the recipient thereof. Typically a sterile water or isotonic formulation is employed. For example, a suitable isotonic solution is phosphate buffered saline or Ringer's solution.

Other examples of pharmaceutically acceptable carriers or diluents are vegetable based oils such as peanut oil, safflower oil, olive oil, cottonseed oil, maize oil, sesame oils arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration. Typically, the carrier or carriers will form from 10% to 99.9% by weight of the compositions.

Formulations may further comprise suitable adjuvants. Adjuvants typically include emollients, emulsifiers, thickening agents, preservatives, bactericides and buffering agents.

For administration as an injectable solution or suspension, non-toxic parenterally acceptable diluents or carriers can include, Ringer's solution, isotonic saline, phosphate buffered saline, ethanol and 1,2 propylene glycol.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof. Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

Compositions may be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which are incorporated herein by reference.

Those skilled in the art will appreciate that the methods and vaccinations contemplated by the present invention may be carried out in conjunction with other therapies or preventative measures for the treatment or prevention of allergic airways diseases or symptoms associated with such diseases. For example, immunotherapy based on sensitisation and challenge with allergens is typically used in the treatment or prevention of asthma. Further, the administration of agonists of various cellular receptors such as TLR-2 and TLR-4 may also be employed. For such combination therapies, each component of the combination therapy may be administered at the same time, or sequentially in any order, or at different times, so as to provide the desired effect. Alternatively, the components may be formulated together in a single dosage unit as a combination product. For example, Streptococcus pneumoniae or fractions, components or constituents thereof can be combined with immunizing or other non-immunizing components to produce a multivalent vaccine or with other medicaments. When administered separately, components may be administered by the same route of administration, although it is not necessary for this to be so.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The present invention will now be described with reference to the following specific examples, which should not be construed as in any way limiting the scope of the invention.

### EXAMPLES

### Example 1

### Mouse Model of Th2-Induced Allergic Airways Diseases

An experimental model of allergic airways disease was established in BALB/c mice (Th2 and IgE responding), based on sensitisation consistency (see below) and subsequent challenge with ovalbumin (OVA). This model was used to determine the effect of Streptococcus pneumoniae infection on the development of allergic airways disease (Example 2) and the effect of immunization with killed Streptococcus pneumoniae on the development and expression of allergic airways disease (Example 3).

Female BALB/c mice (6-8 week old) were obtained from the Central Animal House, University of Newcastle. For the induction of allergic airways disease, mice were sensitised by intrperitoneal injection or intranasal administration of OVA (50 ug; Sigma, Mo. USA). Intraperitoneal sensitisation was carried out at day 0 using OVA in Rehydrogel (1 mg, Reheis, Berkeley Heights, USA) in sterile saline (200 μl) and mice were subsequently challenged by intranasal droplet application of OVA (day 12-15; 10 μg, 50 μl sterile saline). For intranasal sensitisation OVA was administered at day 0 and day 1 without adjuvant.

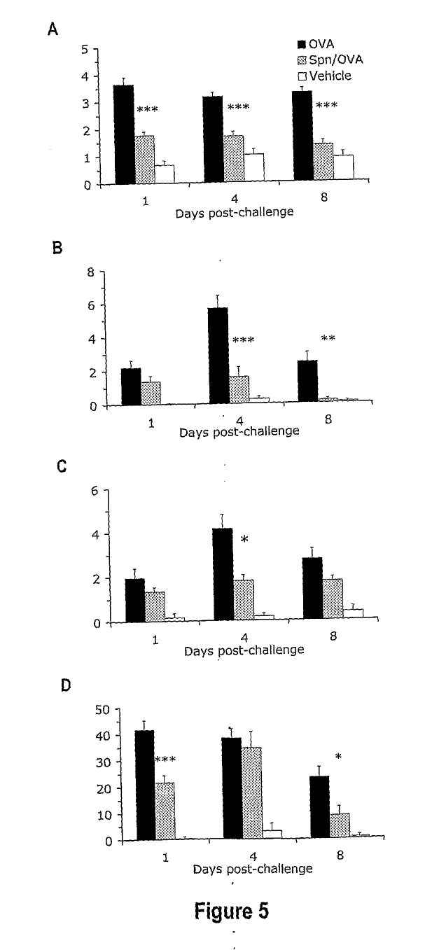

Mice were sacrificed by intraperitoneal injection of sodium pentobarbitone (200 μl) (at day 16 (or up to day 23 in FIG. 5) and inflammatory responses and airways hyperresponsiveness were assessed. Control mice received saline sensitisation and OVA challenge. Mice were held in specific pathogen free conditions, and all procedures were approved by the University of Newcastle Animal Care and Ethics Committee.

For the experiments described in this and the subsequent examples, inflammatory responses and airways hyperresponsiveness were assessed in accordance with the following procedures.

In measuring mediastinal lymph node T cell cytokine production, mediastinal lymph nodes were isolated, homogenised and cultured at 10⁶ cells/well (96 h, 37° C., 5% CO₂), before stimulation as described in Berry et al., 2004 using 200 ug/ml OVA as the stimulant. Cell-free culture supernatants were stored at −20° C. IL-5 and Interferon (IFN)-γ concentrations were determined by ELISA (BD Biosciences, San Diego; USA).

OVA-specific IgG1 and IgG2a antibodies were determined by ELISA as described in Berry et al., 2004 using 40 ug/ml OVA as the capture antigen.

In the assessment of cellular inflammation, blood was collected by cardiac puncture and bronchoalveolar lavage fluid (BALF) was obtained by cannulation of the trachea, and lavage with Hanks balanced salt solution (2×0.7 ml) (see Foster et al., 1996). BALF cell numbers were determined using a hemocytometer. Cells were cytocentrifuged and stained with May-Grunwald Giemsa. Differential cell counts were based on standard morphological characteristics of at least 250 cells/sample. Lungs were fixed, sectioned and stained with chromotrope and light haematoxylin or Periodic Acid-Schiff. Tissue eosinophilia in inflamed peribronchial tissue and epithelial goblet cell hyperplasia were determined as previously described (Foster et al., 1996).

For the measurement of lung function, airways hyperresponsiveness was assessed by determination of airway resistance and dynamic compliance of the airways following challenge with aerosolised methacholine as previously described (Takeda et al., 1997). Anaesthetised, tracheostomised mice were mechanically ventilated (150 strokes/min, 0.18 ml tidal volume), aerosolised methacholine was administered (10 ul) in increasing concentrations (6.25 to 50 mg/ml), and lung function assessed.

Results are presented herein as mean±SEM. Lung function data were analysed by repeated measures one-way ANOVA by comparison of the entire dose-response curve. All other data were analysed by one-way ANOVA with Tukey's post-test.

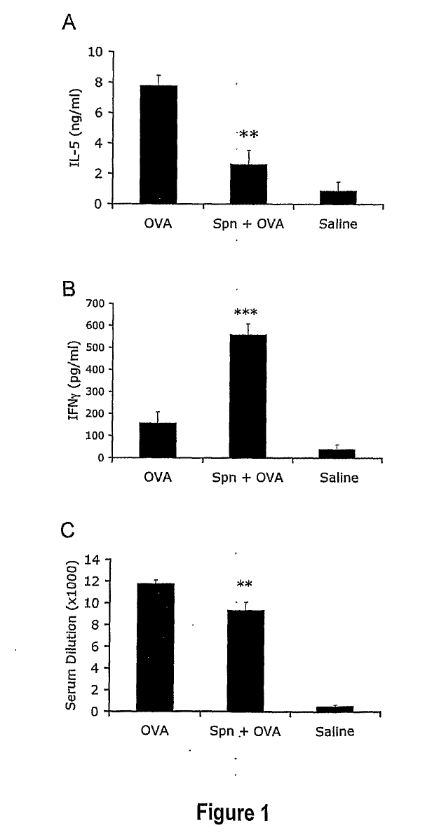

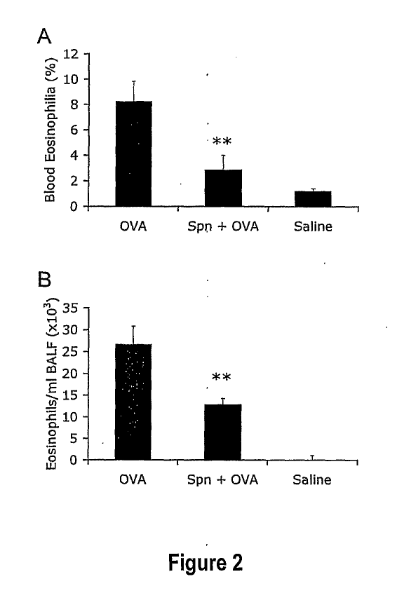

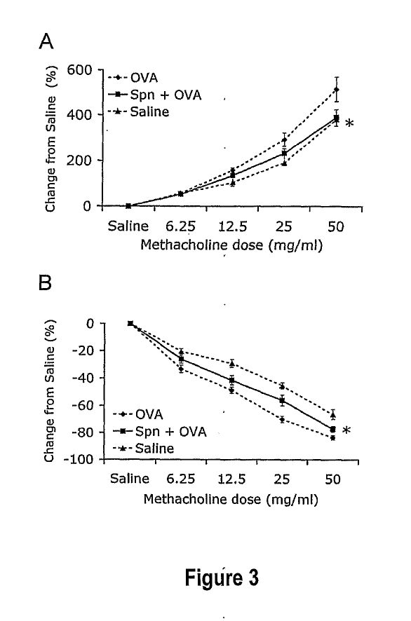

Induction of allergic airways disease by intraperitoneal OVA sensitisation and challenge resulted in significant increases in OVA-specific-IL-5 production by T cells and serum IgG1 antibody levels (see FIG. 1, OVA panel), and induced the recruitment of CD4+ T-lymphocytes (not shown) and eosinophils into the blood and peribronchial spaces (see FIG. 2, OVA panel). This allergic phenotype was associated with the induction of goblet cell hyperplasia in the airways epithelium, and correlated with disease severity. Enhanced airways responsiveness to methacholine directly correlated with the development of allergic airways disease. Airways hyperresponsiveness was characterised by alterations in lung function involving a significant increase and decrease in airways resistance and compliance, respectively (see FIG. 3). Similar results (for eosinophils, mucous secreting cell expression and histopathology) were obtained for intranasal OVA sensitisation (see FIGS. 5 to 7 discussed below in Example 2).

### Example 2

### Effect of Streptococcus pneumoniae Infection on the Development of Allergic Airways Diseases

Streptococcus pneumoniae (Type 3, strain NC012695; available under Accession Number ACTC6303 from the National Collection of Type Cultures, Egham, UK) was provided by Professor J. Kyd of the University of Canberra, Australia. Prior to use, bacteria were stored at −80° C. in tryptone soya broth (TSB, Oxoid Australia) with 5% defibrinated horse blood, 0.5% glucose and 20% glycerol. Bacteria were freshly cultured before each experiment on tryptone soy agar supplemented with 5% blood and 0.5% glucose and incubated for 16 hours (37° C., 5% CO₂). Colonies were harvested, and suspended in phosphate buffered saline (PBS) to yield 2×10⁵ cfu/30 ul. Animals were anaesthetised intravenously with Alfaxan (Jurox, Australia), intubated with a 22-gauge catheter (Hospital Supplies, Australia), and 2×10⁵ cfu Streptococcus pneumoniae was instilled directly into the airway (trachea). OVA sensitisation was either intraperitoneal or intranasal and carried out as described in Example 1.

To assess the effect of a Streptococcus pneumoniae infection on the exacerbation of allergic airways disease, intraperitoneally OVA sensitised mice were infected with live Streptococcus pneumoniae 2 days before OVA challenge. OVA-specific-IL-5 release from T cells was significantly reduced, while IFNγ levels were significantly increased compared to uninfected allergic controls (p<0.01 and p<0.001, FIGS. 1A and B, respectively). OVA-specific serum IgG1 titre, indicative of a Type 2 immune response, was also assessed and found to be significantly reduced by Streptococcus pneumoniae infection (p<0.01, FIG. 1C). IgG2a, which is characteristic of Type 1 responses, was not detected in infected or allergic groups.

In sensitised mice infected with Streptococcus pneumoniae before OVA challenge the number of eosinophils in both the blood and BALF were significantly reduced compared to uninfected allergic controls (p<0.01, FIG. 2). Importantly, the number of eosinophils in the blood decreased to similar level to those of the saline control group (FIG. 2A). However, there was no difference in eosinophil or goblet cell numbers in the peribronchial tissue (data not shown.)

A significantly smaller change in resistance and compliance over baseline, which approached background levels, was observed in mice infected with Streptococcus pneumoniae before OVA challenge compared to uninfected allergic controls (p<0.05, FIGS. 3A and B).

To examine the influence of timing of infection relative to antigen (OVA) exposure on the development of allergic airways disease, mice were infected with Streptococcus pneumoniae either before (resolved infection) or during (concurrent infection) intraperitoneal sensitisation to OVA. In the case of resolved infection, administration of Streptococcus pneumoniae was carried out 10 days prior to OVA sensitisation, whereas for concurrent infection, Streptococcus pneumoniae was administered at the same time as OVA sensitisation.

The inventors have previously shown the Streptococcus pneumoniae infection 10 days before sensitisation allowed bacterial clearance and recovery from inflammation before antigen (OVA) exposure whilst infection concurrent with sensitisation ensured that maximal immunomodulatory effects of infection were induced because sensitisation occurred in a background of peak immune responses to Streptococcus pneumoniae infection (Preston et al., 2004).

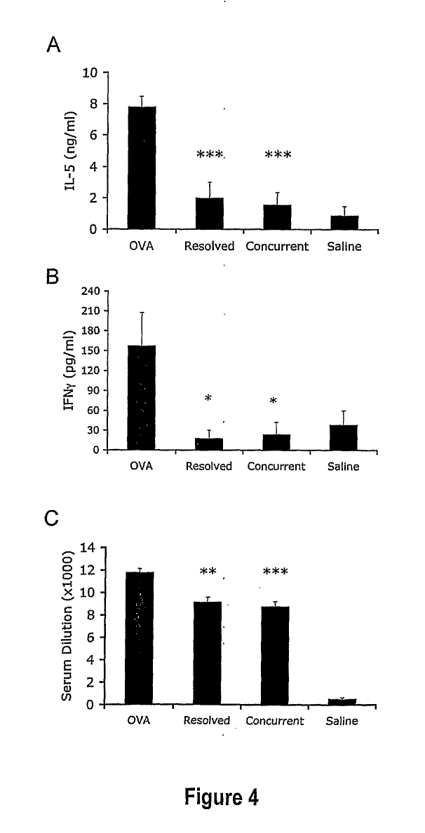

Ex vivo stimulation of T cells with OVA from mice with a resolved or concurrent Streptococcus pneumoniae infection at the time of intraperitoneal OVA sensitisation demonstrated that infection significantly inhibited the production of both OVA-specific-IL-5 (p<0.001, FIG. 4), and -IFNγ (p<0.05, FIG. 4B). Indeed IFNγ levels were below the limit of detection. Both resolved and concurrent Streptococcus pneumoniae infection also significantly reduced the serum OVA-specific IgG1 titre compared to uninfected allergic controls (FIG. 4C). Again, the type 1 antibody IgG2a was not detected.

For the experiments described below in relation to the results shown in FIGS. 5 to 7, OVA sensitisation was intranasal. Similar results were obtained for intraperitoneal sensitisation (FIGS. 1 to 4 and data not shown).

Streptococcus pneumoniae infection during intranasal allergic sensitisation significantly suppressed allergic inflammation of the lung and was sustained for at least 8 days. Mice were infected with Streptococcus pneumoniae at the same time as intranasal sensitisation with OVA and eosinophilia in the blood, BALF and peribronchial tissue, goblet cell hyperplasia of the large airways and histopathology were assessed. Analysis was conducted 1, 4 and 8 days after the final OVA challenge. The percentage of eosinophils in the blood was significantly decreased 1 day after OVA challenge (P<0.001, FIG. 5A), and returned to background (PBS vehicle) control levels within 4 days in the Streptococcus pneumoniae infected group (Spn/OVA) compared to uninfected allergic controls. The number of eosinophils in the BALF was significantly decreased following Streptococcus pneumoniae infection compared to the uninfected allergic controls within 4 days after challenge (FIG. 5B). Streptococcus pneumoniae infection also suppressed the accumulation of eosinophils in tissue surrounding the airways, although results only reached statistical significance at 4 days after OVA challenge compared to uninfected allergic controls (p<0.01, FIG. 5C).

Furthermore, infection with Streptococcus pneumoniae significantly suppressed goblet cell hyperplasia after OVA challenge (p<0.001 1 day and p<0.05 8 days after challenge, FIG. 5D). The number of mucus-positive cells was reduced to control levels by 8 days post-challenge. Lung histopathology was not significantly altered by Streptococcus pneumoniae infection during OVA sensitisation compared to uninfected allergic controls (data not shown). The maximal inflammatory responses to OVA, as well as detectable differences between infected and uninfected groups were observed at 4 days after challenge, therefore analysis was performed at this time point in all further experiments using intranasal sensitisation.

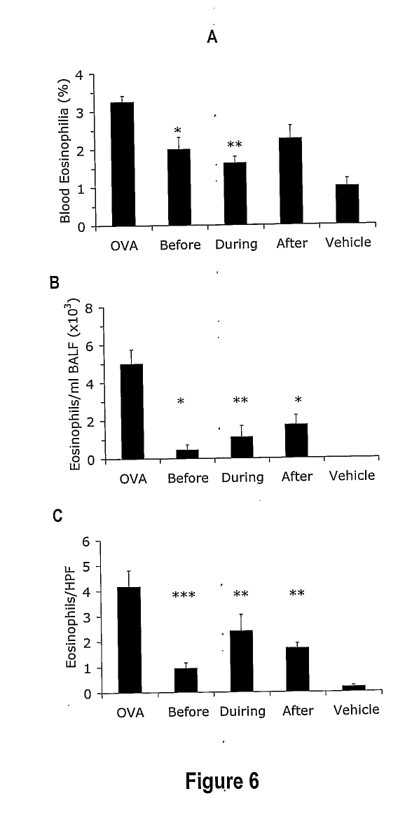

Streptococcus pneumoniae infection suppressed the development and exacerbation of allergic lung inflammation regardless of the timing of infection relative to intranasal OVA sensitisation. This demonstrates that Streptococcus pneumoniae infection may have inhibitory effects on allergic airway disease that is induced directly in the lung. To examine the influence of timing of Streptococcus pneumoniae infection relative to OVA sensitisation on the development of eosinophilic inflammation, mice were infected before, during or after OVA sensitisation and eosinophilia in blood, BALF and tissue were assessed 4 days after the final OVA challenge. Infection with Streptococcus pneumoniae before or during OVA sensitisation significantly reduced blood eosinophilia compared to uninfected allergic controls (P<0.05 and p<0.01, respectively, FIG. 6A). Infection after sensitisation induced a non-significant trend towards a reduction in blood eosinophils. Eosinophil accumulation in the BALF and peribronchial tissue was also significantly reduced following Streptococcus pneumoniae infection before, during or after intranasal OVA sensitisation (FIGS. 6B and C).

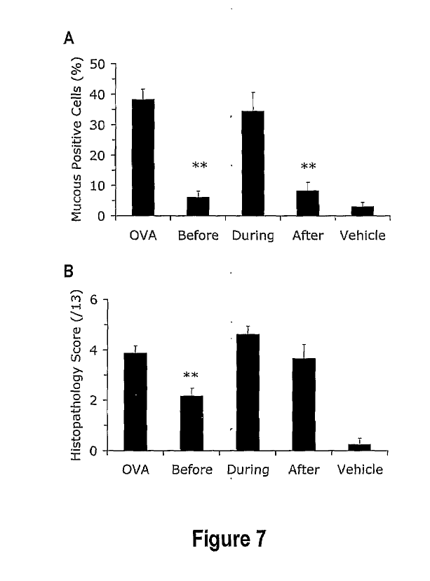

To further explore the effect of timing of Streptococcus pneumoniae infection on respiratory inflammation, goblet cell hyperplasia and lung tissue histopathology were also characterised. Streptococcus pneumoniae infection before or after OVA sensitisation significantly inhibited goblet cell hyperplasia (p<0.01. FIG. 7A). However, Streptococcus pneumoniae infection during OVA sensitisation had no effect on the number of mucus-positive cells in the airway epithelium Streptococcus pneumoniae infection before, but not during or after OVA sensitisation significantly decreased the severity of histopathological inflammation in the lungs (p<0.01, FIG. 7B).

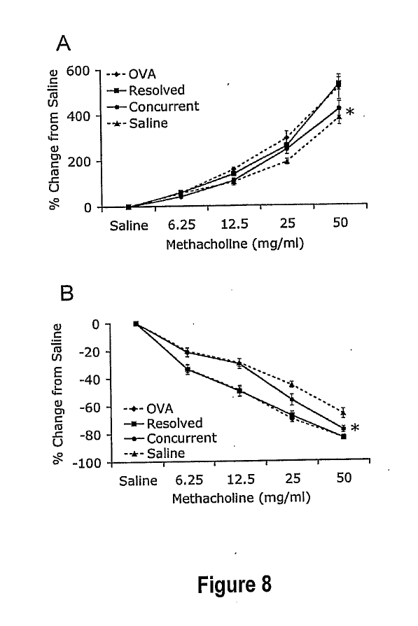

Resolved Streptococcus pneumoniae infection before intraperitoneal OVA sensitisation had no significant affect on airway resistance or compliance in response to methacholine challenge. However, mice infected with Streptococcus pneumoniae during sensitisation had a significantly smaller change in both airway resistance and compliance over baseline, compared to uninfected allergic controls (p<0.05, FIG. 8).

The above results suggest that, in general, Streptococcus pneumoniae infection suppresses key features of allergic airways diseases regardless of the timing of the infection.

### Example 3

### Effect of Vaccination Using Killed Streptococcus pneumoniae on the Development of Allergic Airways Diseases

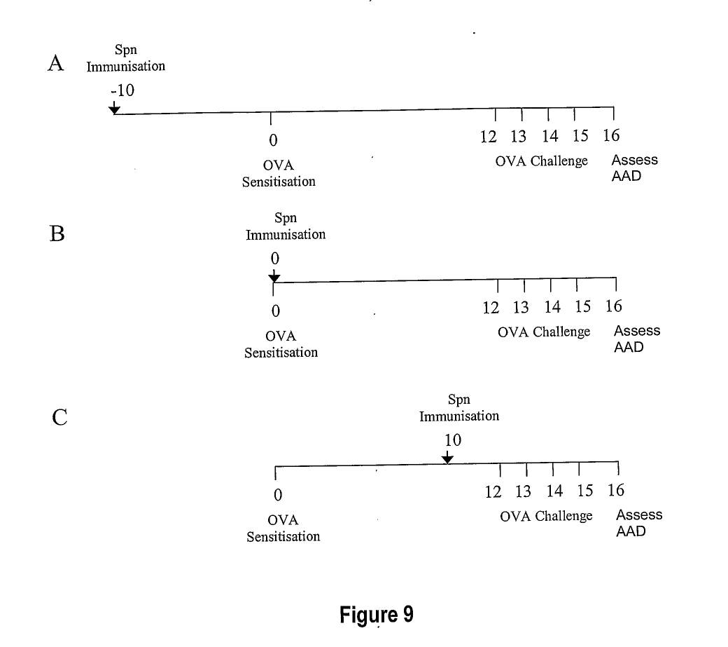

In order to investigate the potential of Streptococcus pneumoniae immunisation to inhibit the development and expression of allergic airways disease, and to differentiate between the modulation of allergic airways disease by an active Streptococcus pneumoniae infection and antigen exposure, the inventors assessed the affect of killed whole Streptococcus pneumoniae inoculation prior to OVA challenge on allergic airways disease. Experimental protocols were established to examine three different models, as illustrated schematically in FIG. 9. In the first, mice were immunised with whole killed Streptococcus pneumoniae 10 days prior to intraperitoneal OVA sensitisation (FIG. 9A). In the second, Streptococcus pneumoniae immunisation was concurrent with intraperitoneal OVA sensitisation (FIG. 9B). In the third, immunisation with Streptococcus pneumoniae occurred 10 days after intraperitoneal OVA sensitisation (FIG. 9C).

Streptococcus pneumoniae type 3, strain NC012695, was cultured, harvested and suspended in PBS as described in Example 2. Ethanol-killed Streptococcus pneumoniae were then prepared as described in Malley et al. (2001), and stored at −80° C. until required. Mice were sensitised intraperitoneally with OVA at day 0 (as described in Example 1). At day-10, 0 or 10 mice were inoculated with killed Streptococcus pneumoniae (3×, every 12 hours) as described by Bergeron et al. (1998). This ensured that Streptococcus pneumoniae antigens were present in lungs for the equivalent period as for live Streptococcus pneumoniae infection (Example 2).

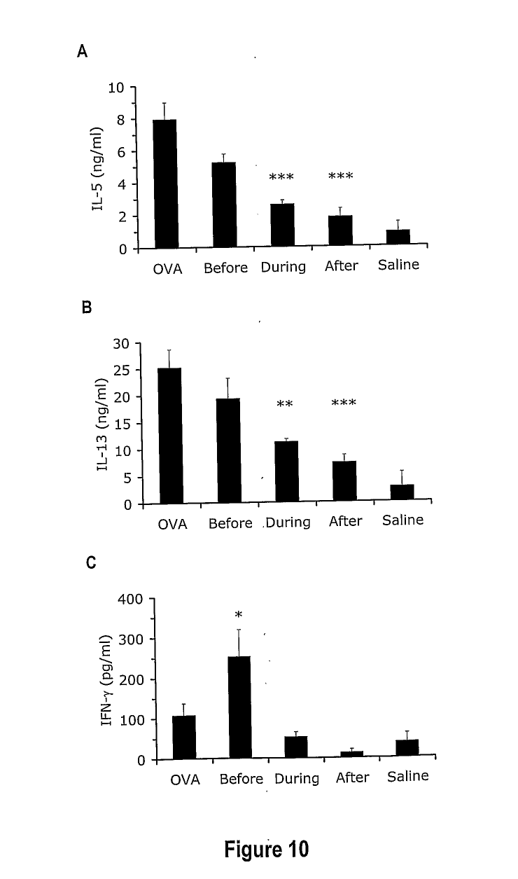

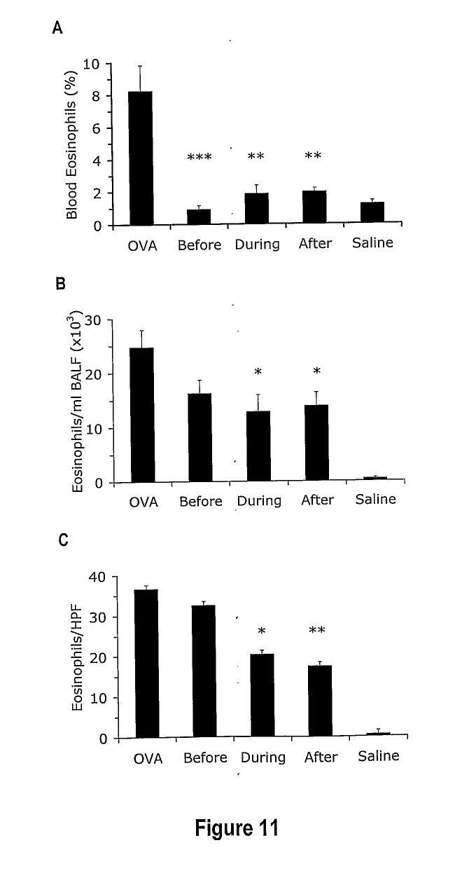

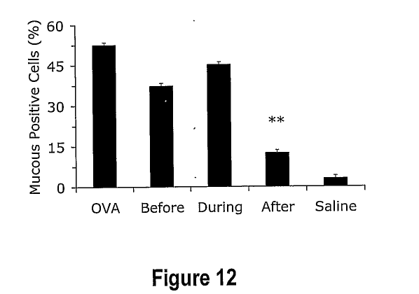

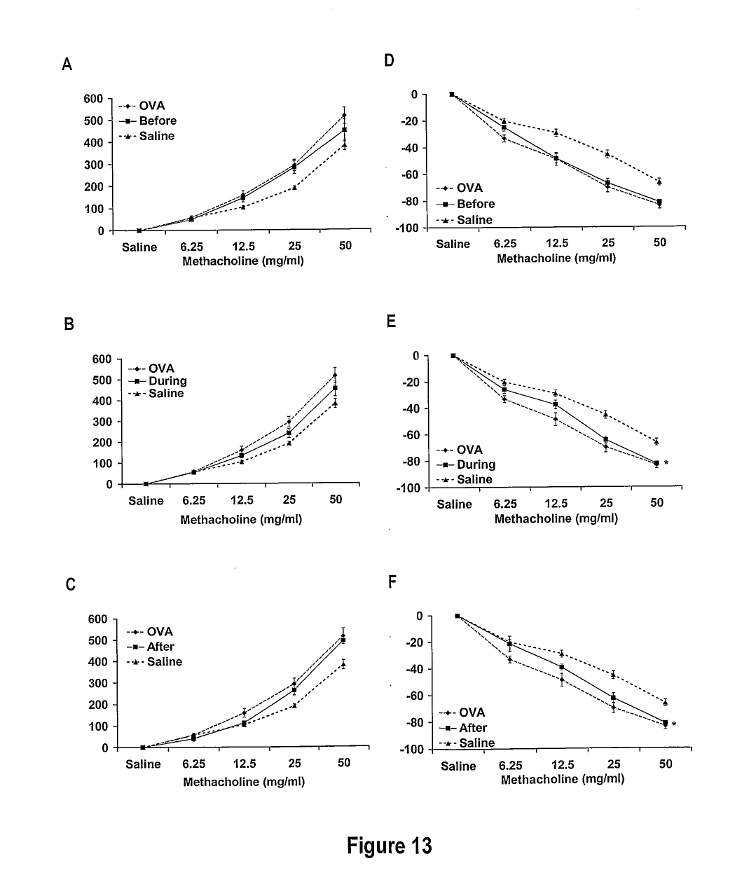

To assess the effect of immunisation with killed Streptococcus pneumoniae on the development of allergic airways disease mice were immunised with killed Streptococcus pneumoniae 10 days before intraperitoneal OVA sensitisation. Immunisation caused a trend towards a decrease in OVA-specific IL-5 and IL-13 release (Before panels of FIGS. 10A and 10B, respectively) and a significant increase in IFNγ secretion (p<0.05, FIG. 10C, Before panel) from T cells compared to uninfected allergic controls. Immunisation before OVA sensitisation also caused a significant decrease in eosinophil numbers in the blood (p<0.001, FIG. 11A, Before panel) and a trend towards a decrease in eosinophil numbers in the BALF and lung tissue (Before panels of FIGS. 11B and 11C, respectively) compared to uninfected allergic controls. Importantly, the number of eosinophils in the blood decreased to similar level to those of the saline control group. Immunisation before OVA sensitisation also caused a trend towards a decrease in the numbers of mucous secreting cells around the airways (FIG. 12, Before panel) compared to uninfected allergic controls. Immunisation before OVA sensitisation had little effect on changes in resistance and compliance (FIGS. 13A and 13D) compared to uninfected allergic controls.

To examine the influence of timing of immunisation relative to antigen (OVA) exposure on the development and exacerbation of allergic airways disease, mice were immunized with killed Streptococcus pneumoniae during or after intraperitoneal sensitisation with OVA. Immunisation caused a significant decrease in OVA-specific IL-5 and IL-13 release (p<0.01 to p<0.001, FIGS. 10A and 10B, During and After panels, respectively) and a trend towards a decrease in IFNγ secretion (FIG. 10C, During and After panels) from T cells compared to uninfected allergic controls. Immunisation during or after OVA sensitisation also caused a significant decreased in eosinophil numbers in the blood, BALF and lung tissue (p<0.05 to p<0.01, FIGS. 11A, B and C, During and After panels, respectively) compared to uninfected allergic controls. Importantly, the number of eosinophils in the blood decreased to similar level to those of the saline control group. Immunisation during OVA sensitisation also caused a trend towards a decrease in the numbers of mucous secreting cells around the airways (p<0.01, FIG. 12, During panel) while immunisation after OVA sensitisation caused a significant decrease in the numbers of mucous secreting cells (p<0.01, FIG. 12, After panel) compared to uninfected allergic controls. Immunisation during or after OVA sensitisation caused a trend toward a decrease in airway resistance (FIGS. 13B and 13C) and a significant increase in compliance (p<0.05, FIGS. 13E and 13F) compared to uninfected allergic controls.

These results, taken together with those of Example 2, indicate that it is the effect of Streptococcus pneumoniae antigens, rather than an inflammatory response to live Streptococcus pneumoniae infection that is driving the inhibition of allergic airways disease.

### Example 4

### T Cell Numbers in Response to Streptococcus pneumoniae Infection in a Mouse Model of Allergic Airways Disease

Regulatory T cells (Treg cells) are known to suppress the development of allergic inflammation and airway hyperresponsiveness (Kearley et al., 2005). To determine the effect of Streptococcus pneumoniae infection on Treg cell numbers in the mouse model of allergic airways disease established in Example 1, mice were infected with Streptococcus pneumoniae at the same time as being intraperintoneally sensitised with OVA. OVA sensitisation was carried out as described in Example 1. The mice were challenged intranasally with OVA 12-15 days later. Cells from mediastinal lymph nodes from these mice were cultured for 96 hr either in the presence or absence of OVA (200 μg/ml) prior to staining and analysis by flow cytometry as described previously (Zhao et al., 2007). Staining was for the T cell markers CD4 and CD25, the co-expression of which is considered indicative of Treg cells, and the Treg cell marker Foxp3.

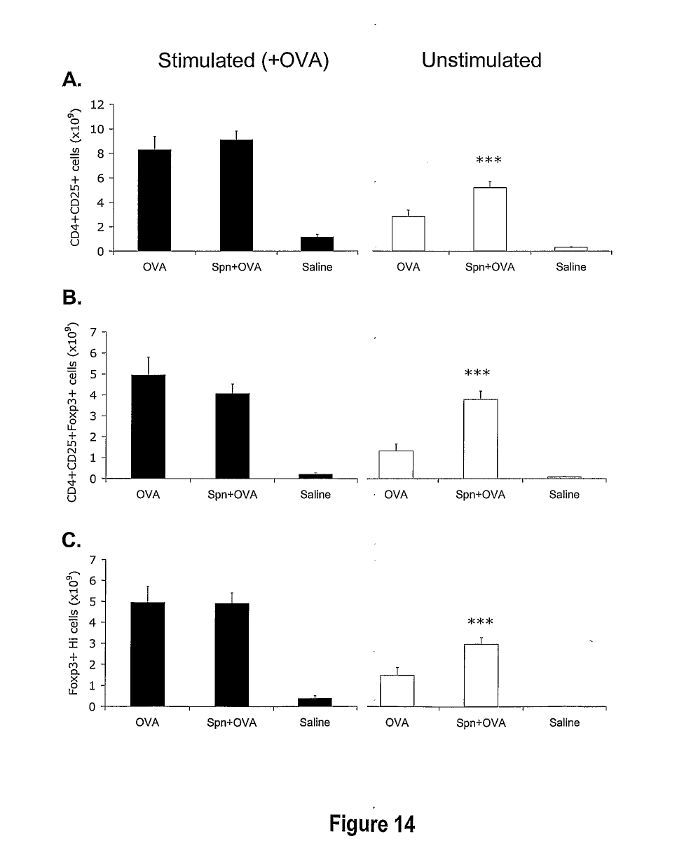

As shown in FIG. 14A, the number of CD4+CD25+ cells was observed to be significantly higher in mice infected with Streptococcus pneumoniae and sensitised with OVA (Spn+OVA group) when compared to uninfected mice (OVA group). This was observed in cells not stimulated with OVA prior to staining. Similarly, additional staining for the T reg-specific transcription factor Foxp3 also revealed a higher number of this population of cells in the infected and sensitised (Spn+OVA) group (FIG. 14B). The number of cells determined to be expressing high levels of Foxp3 (Foxp3+ Hi) were also elevated in the Spn+OVA group (FIG. 14C).

When stimulated with OVA prior to staining and analysis, the number of Treg cells were comparable between the infected and uninfected groups. This suggests that stimulation of cultures with OVA stimulates another population of OVA-specific cells that may also increase the number of Treg cells.

To determine the role of the Treg cells in the Spn+OVA group, CD4+ cells from the OVA group were cultured with unstimulated Treg cells isolated from the Spn+OVA group, In a T cell proliferation assay using CFSE staining (following similar protocols to those described in Venken et al., 2007), the effect of Treg cells on the proliferation on the CD4+ population was examined.

CD4+CD25− and CD4+CD25+ cell populations from mouse spleens, of the OVA and Spn+OVA groups respectively, were isolated by indirect magnetic cell sorting. CD4+CD25− cells were stained with CFSE and cultured 2×10⁴ cells per well with 1×10⁵ mitomycin treated splenocytes and OVA. 2×10⁴ CD4+CD25+ cells were added to half of these wells and cultured for 96 hr. Subsequently, cells were stained for CD4+ and analysed via flow cytometry.

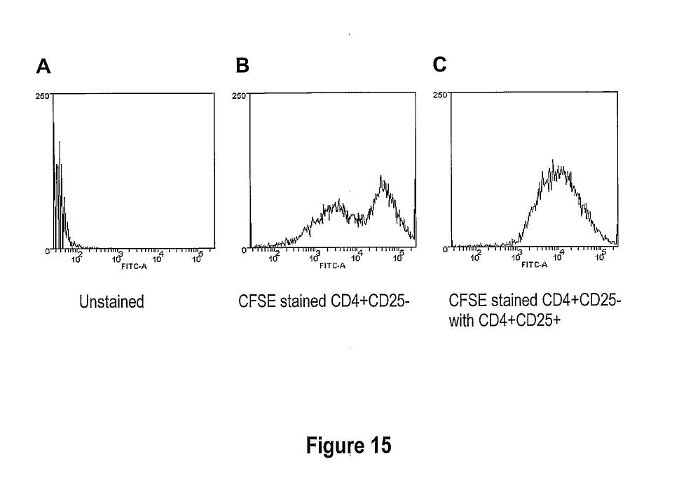

Cells expressing both CD4+ and CFSE were gated and the results shown in FIG. 15. CD4+CD25− cells proliferate as shown by the shift to the left (FIG. 15B). When CD4+CD25+ cells were added, this shift was not observed. Rather, the proliferation of the CD4+CD25− cells was suppressed (FIG. 15C). Thus, Treg cells induced upon Streptococcus pneumoniae infection in the Spn+OVA group suppress the CD4+ T helper cell response to OVA.

In this example, it has been shown that there are higher numbers of Treg cells in the Spn+OVA group when compared to the uninfected allergic (OVA) group. This alone suggests that Treg cells play an important role in the modulation of the Th2 response. Furthermore, it has been shown that these Treg cells can suppress the allergen-specific CD4 T helper cell response. Whilst not wishing to be bound by any one particular theory, since intervention of the OVA model with Streptococcus pneumoniae reduces the Th2 response, it is proposed that this may be one mechanism that is involved in modulation of the response allowing for the reduction of allergic airways disease.

### REFERENCES

Bergeron, Y et al. (1998) Antimicrob Agents Chemother 42:2527-33.
Berry, L J et al. (2004) Infect Immun 72:1019-28.
Carlsen, K-H (2004) Paediatric Respiratory Reviews 5:45-51.
Dziarski, R and D Gupta (2005) Infect Immun 73:5212-6.
Foster, P S et al. (1996) J Exp Med 183:195-201.
Jones, P D et al. (2000) Med Hypotheses 55:40-42.
Kearley J et al (2005) J Exp Med 202:1539.
Malley, R et al. (2001) Infect. Immun. 69:4870-4873.
Ogunniyi A D et al. (2000) Infect Immun 68:3028-33.
Ogunniyi A D et al. (2001) Infect Immun 69:5997-6003.
Preston, J A et al. (2004) Eur Respir J 23:224-31.
Salisbury, D M, and N T Begg. (1996), Immunisation against infectious diseases, HMSO, London.
Schroder, N W et al. (2003) J Biol Chem 278:15587-94.
Takeda, K et al. (1997) J. Exp Med 183:449-54.
Talbot, T R et al. (2005) N Engl J Med 352:2082-2090.
Venken K et al. (2007) J Immunol Meth 322:1-11
Zhao L et al. (2007) Leukoc Biol 81:1386-94

1. A method for the treatment or prevention of an allergic airways disease or suppression of onset of an allergic airwavs disease in a subject, the method comprising administering to the subject a composition comprising Streptococcus pneumoniae or one or more antigens derived therefrom.

2. The method of claim 1 wherein the composition is a therapeutic or prophylactic vaccine.

3. The method of claim 1 wherein the composition comprises killed or attenuated Streptococcus pneumoniae.

4. The method of claim 1 wherein the one or more antigens comprise cellular fractions, components or constituents.

5. (canceled)

6. The method of claim 4 wherein the one or more antigens comprise cell wall peptidoglycan, lipoteichoic acid, cell wall polysaccharides or proteins, and/or capsular polysaccharides or proteins.

7. The method of claim 1 wherein the allergic airways disease is selected from asthma, asthma exacerbations, eosinophilic bronchitis, allergic rhinitis, chronic cough, sinusitis, angioedema, urticaria, chronic obstructive pulmonary disease, conjunctivitis and hay fever.

8. The method of claim 7 wherein the asthma is chronic asthma or acute asthma.

9. (canceled)

10. The method of claim 1 wherein the composition is administered in conjunction with one or more suitable adjuvants designed to improve the delivery or efficacy of the composition.

11. (canceled)

12. The method of claim 1 wherein protective immunity against an allergic airways disease is induced in the subject.

13. A method for the suppression of an allergic immune response in a subject, the method comprising administering to the subject a composition comprising Streptococcus pneumoniae or one or more antigens derived therefrom.

14. (canceled)

15. The method of claim 13 wherein the allergic immune response is a Th2 immune response.

16. The method of claim 13 wherein the allergic immune response is associated with eosinophilia, mucous secreting cell expression, airway hyperresponsiveness and/or a Th2-mediated disease.

17. The method of claim 16 wherein the eosinophilia is peripheral eosinophilia or tissue eosinophilia.

18-19. (canceled)

20. A composition for use in the treatment or prevention of allergic airways diseases or the suppression of onset of an allergic airways disease in a subject the composition comprising Streptococcus pneumoniae or one or more antigens derived therefrom.

21. The composition of claim 20 wherein the composition is a therapeutic or prophylactic vaccine.

22. The composition of claim 20 wherein the composition comprises killed or attenuated Streptococcus pneumoniae.

23. The composition of claim 20 wherein the one or more antigens comprise cellular fractions, components or constituents.

24. (canceled)

25. The composition of claim 23 wherein the one or more antigens comprise cell wall peptidoglycan, lipoteichoic acid, cell wall polysaccharides or proteins, and/or capsular polysaccharides or proteins.

26. The composition of claim 20 wherein the allergic airways disease is selected from asthma, asthma exacerbations, eosinophilic bronchitis, allergic rhinitis, chronic cough, sinusitis, angioedema, urticaria, chronic obstructive pulmonary disease, conjunctivitis and hay fever.

27. The composition of claim 26 wherein the asthma is chronic asthma or acute asthma.

28-31. (canceled)
